(19) United States
(12) Patent Application Publication
(10) Pub. No.: US 2014/0257014 A1
(43) Pub. Date: Sep. 11, 2014

(54) TREADMILL AUTOMATED DOSING USER INTERFACE

(71) Applicant: MEDRAD, INC., Indianola, PA (US)

(72) Inventor: Larry McCUTCHAN, Allison Park, PA (US)

(73) Assignee: MEDRAD, INC., Indianola, PA (US)

(21) Appl. No.: 13/789,535

(22) Filed: Mar. 7, 2013

(51) Int. Cl. A61N 5/10

(52) U.S. Cl. CPC A61N 5/1064; A61N 5/1001
USPC 600/4

(57) ABSTRACT

Methods and systems for dispensing a radionuclide are disclosed. A processor may receive one or more physiological signals for a patient from one or more physiological parameter sensors. The processor may compare the physiological signals with one or more threshold values. The processor may cause an injector device to dispense the radionuclide in response to the one or more physiological signals meeting or exceeding the one or more threshold values. The threshold values may be determined at least in part based on one or more characteristics of the patient.

### BACKGROUND

The present disclosure relates to methods and systems for automating dosing of a radionuclide. More particularly, the present disclosure relates to methods and systems for automatic dosing of a radionuclide to a patient for an imaging procedure, such as myocardial perfusion imaging, based on physiological information received from the patient and/or other information.

Myocardial perfusion imaging is a diagnostic method used to detect and characterize coronary artery disease. Perfusion imaging makes use of, for example, radionuclides to identify areas of insufficient blood flow in a patient. In myocardial perfusion imaging, the progress of a radionuclide into the myocardium is measured at rest and under a condition of cardiac stress, such as physical exertion or chemically induced stress, to estimate blood flow. A comparison is performed between the two blood flow measurements to determine whether occlusions are present.

When examining the patient's blood flow under the cardiac stress condition, the radionuclide should be infused at a time at which the patient's heart is under sufficient stress. However, infusing the radionuclide at an appropriate time has been left to approximation and guess work using conventional techniques.

### SUMMARY

The invention described in this document is not limited to the particular systems, methodologies or protocols described, as these may vary. The terminology used herein is for the purpose of describing particular embodiments only, and is not intended to limit the scope of the present disclosure.

It must be noted that as used herein and in the appended claims, the singular forms “a,” “an,” and “the” include plural reference unless the context clearly dictates otherwise. Unless defined otherwise, all technical and scientific terms used herein have the same meanings as commonly understood by one of ordinary skill in the art. As used herein, the term “comprising” means “including, but not limited to.”

In an embodiment, a system for dispensing a radionuclide may include a processor, one or more physiological parameter sensors in operable communication with the processor, an injector device in operable communication with the processor, and a non-transitory, computer-readable storage medium in operable communication with the processor. The computer-readable storage medium contains one or more programming instructions that, when executed, cause the processor to receive one or more physiological signals from the one or more physiological parameter sensors, compare the one or more physiological signals with one or more threshold values, and cause the injector device to dispense a radionuclide in response to the one or more physiological signals meeting or exceeding the one or more threshold values.

In an embodiment, a method of dispensing a radionuclide may include receiving, by a processor, one or more physiological signals from one or more physiological parameter sensors, comparing, by the processor, the one or more physiological signals with one or more threshold values, and automatically dispensing, by an injector device in communication with the processor, a radionuclide into a patient in response to the one or more physiological signals meeting or exceeding the one or more threshold values.

### BRIEF DESCRIPTION OF THE DRAWINGS

FIG. 1 depicts a block diagram of an illustrative system for dispensing a radionuclide according to an embodiment.

FIG. 2 depicts a flow diagram of an illustrative method of dispensing a radionuclide according to an embodiment.

FIG. 3 depicts a block diagram of illustrative internal hardware that may be used to contain or implement program instructions according to an embodiment.

### DETAILED DESCRIPTION

As set forth more fully throughout the present disclosure, a processor may include an electronic device, such as, for example, a computer, a server or components thereof. The processor may be operated by a health care professional for use in assisting with an imaging process, such as a myocardial perfusion imaging process. The processor may generally contain a non-transitory memory or other storage device for housing programming instructions, data or information regarding one or more applications, data or information regarding user information and/or the like. The data may be stored in a database in the memory or other storage device. The processor may be in operable communication with one or more other devices. The processor may include a stand-alone processor. The processor may be co-located with one or more other devices or remotely located from other devices. Alternately, the processor may be integrated into a larger device with one or more other sub-components that perform a variety of features, such as those described in this disclosure.

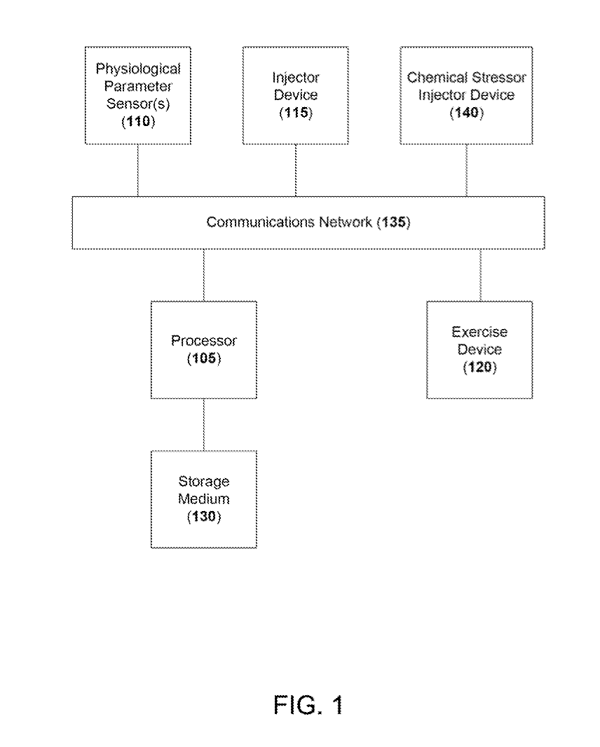

FIG. 1 depicts a block diagram of an illustrative system for dispensing a radionuclide according to an embodiment. As shown in FIG. 1, the system may include a processor 105, one or more physiological parameter sensors 110, and an injector device 115. The processor 105 may be in operable communication with each of the one or more physiological parameter sensors 110 and the injector device 115. For example, the processor 105 may receive information from at least one of the one or more physiological parameter sensors 110 and provide information to the injector device 115 via one or more communication interfaces. In an embodiment, the processor 105 may additionally or alternately receive information from the injector device 115 or one or more other devices. In an embodiment, the processor 105 may additionally or alternately provide information to one or more other devices. For example, the system may include an exercise device 120 from which telemetric signals may be received and/or to which regimen signals may be provided. For example, telemetric signals may identify an amount of resistance that an exercise device 120 is applying, a speed at which an exercise device is operating, and/or a level of incline (inclination) at which an exercise device is set. Conversely, regimen signals may provide updated operational values for the exercise device. The above-disclosed embodiments are merely illustrative; those skilled in the art will recognize that the system may include additional or fewer devices without departing from the scope of this disclosure.

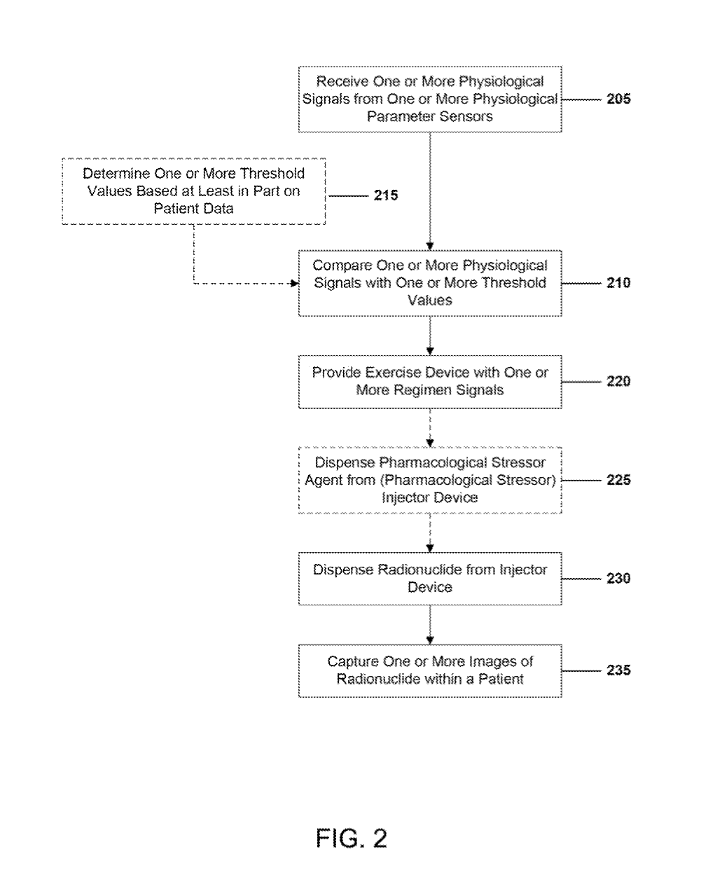

The processor 105 may be in operable communication with a non-transitory storage medium 130. In some embodiments, the processor 105 may be a server. In embodiments where more than one processor 105 is used, each processor may operate independently of the other processors or may operate in an array-type configuration where the processors act as a single unit. In an embodiment, the processor 105 may be in operable communication with a plurality of storage media 130. The one or more storage media 130 may be any type of electronic storage device now known or later developed, including, but not limited to, hard disk drives, solid state drives, removable media drives, optical disc drives, individual memory devices, such as RAM and ROM, and the like, or combinations thereof. In some embodiments, the one or more storage media 130 may be controlled by the processor 105. At least one storage medium 130 may contain one or more programming instructions that, when executed, cause the processor 105 to perform a method of dispensing a radionuclide, such as the one discussed in more detail in reference to FIG. 2 below.

The one or more physiological parameter sensors 110 may include any sensor capable of detecting physiological information from a patient. The one or more physiological parameter sensors 110 may include, without limitation, an electrocardiographic (EKG) monitor, a blood pressure monitor, and/or a pulse oximeter. The one or more physiological parameter sensors may provide one or more physiological signals corresponding to physiological information of a patient to the processor 105 in the form of signals transmitted via a communication network 135 as described below. For example, the one or more physiological signals may include, without limitation, one or more of a blood pressure signal, a heart rate signal, a pulse oximeter signal, an S-T interval signal, and a Q-R-S waveform signal. The processor 105 may compare the one or more physiological signals with one or more threshold values. In an embodiment, a threshold value may be a constant value associated with particular physiological information. In an embodiment, a threshold value may be associated with a value for a slope of a physiological signal with respect to time. In an alternate embodiment, a rest cardiac rate may be compared with a stress cardiac rate. Alternate and/or additional threshold values may be used and/or alternate comparisons may be made within the scope of this disclosure. Further information regarding the use of physiological parameter sensors according to embodiments is provided below.

The injector device 115 is a device configured to inject, dispense, infuse or otherwise provide a substance into or to a patient. In an embodiment, the injector device 115 may inject, dispense or infuse a radionuclide into a patient. In an embodiment, the radionuclide may be technetium-99m (^{99m}Tc). In an alternate embodiment, the radionuclide may be rubidium-82 (⁸²Rb). In still other embodiments, the radionuclide may be thallium-201, iodine-123 (¹²³I), or iodine-131 (¹³¹I). In another embodiment, the radionuclide may be ¹⁸F-deoxyglucose (FDG). In alternate embodiments, the radionuclide may be ¹³N-ammonia or ¹¹C-choline. Additional and/or alternate radionuclides may be used within the scope of this disclosure, including any radionuclides used for a single-photon emission computed tomography (SPECT) procedure, a positron emission tomography (PET) procedure or any other medical imaging procedure. An exemplary device used for a PET procedure is the Intego™ PET Infusion System designed and sold by the MEDRAD business unit of Bayer HealthCare (Indianola, Pa.). Descriptions of exemplary SPECT and PET devices are also found in U.S. Provisional Application No. 61/656,716 to Kaintz et. al.; U.S. Pat. No. 8,198,599 to Bouton et al., U.S. Pat. No. 6,767,319 to Reilly et al., PCT Patent Application Publication WO 2004/004787 to Van Naemen et al., EPO Patent Application Publication EP 1,616,587 to Buck, and U.S. Patent Application Publication No. 2008/0177126 to Tate et al., each of which is hereby incorporated by reference in its entirety.

In an embodiment, the injector device 115 or a separate pharmacological stressor injector device 140 may be used to inject a pharmacological stressor agent as part of a myocardial perfusion imaging process or other imaging process. In an embodiment, the injector device 115 and the pharmacological stressor injector device 140 may be part of the same device. In an embodiment, the pharmacological stressor agent may be adenosine. In an alternate embodiment, the pharmacological stressor agent may be dobutamine. In still other embodiments, the pharmacological stressor agent may be regodenoson, dipyridamole, or any vasodilator and/or drug that interacts with an A1 or A2A receptor. Additional and/or alternate radionuclides may be used within the scope of this disclosure.

The exercise device 120 may be used as part of a myocardial perfusion imaging process or other imaging process. A patient may use the exercise device 120 to provide stress to the patient's cardiovascular system. The exercise device 120 may include, for example and without limitation, a treadmill, an exercise bicycle, a stair climber or an elliptical aerobic machine. Additional and/or alternate exercise devices 120 may be used within the scope of this disclosure. In some embodiments, an exercise device 120 may not be used, for example, if the patient is incapable of exercising. In such embodiments, a pharmacological stressor agent, such as is described above, may be used alone to stress the patient's cardiovascular system.

In an embodiment, the exercise device 120 may have a pre-programmed protocol for developing cardiac stress in a patient by exercising the patient. The protocol may be designed to induce stress in a patient by varying, for example, the speed, resistance and/or inclination of the exercise device 120. In an embodiment, the protocol may be determined based at least in part on one or more characteristics of the patient, such as the patient's age, gender, height, weight and/or body mass index. In an embodiment, the pre-programmed protocol may be modified based on one or more physiological signals received by the processor 105 from the physiological parameter sensors 110.

A communications network 135 may serve as an information highway interconnecting the other illustrated components. The communications network 135 is not limited by this disclosure, and may include any communications network now known or later developed. The communications network 135 may utilize any suitable data communication, telecommunication, wired, wireless, and/or near-field interface or the like. The communications network 135 may be used to connect any number of devices, systems or components, and may further use any number of communications links. For example, the communications network 135 may use one or more of a local area network (LAN), a wide area network (WAN), a wireless local area network (WLAN), the internet, a cellular network, a paging network, a private branch exchange (PBX) and/or the like.

The processor 105 may be coupled to the communications network 135 via a communications link, such as, for example, a wired link, a wireless link or any combination thereof. Furthermore, each of the one or more physiological parameter sensors 110, the injector device 115, the exercise device 120 (if any) and/or the pharmacological stressor injector device 140 (if any) may be coupled to the communications network 135 via a communications link, such as, for example, a wired link, a wireless link or any combination thereof.

FIG. 2 depicts a flow diagram of a method of dispensing a radionuclide according to an embodiment. As shown in FIG. 2, a processor may receive 205 one or more physiological signals from one or more physiological parameter sensors. The one or more physiological parameter sensors may be used to receive physiological information from a patient. For example, the one or more physiological parameter sensors may be in contact with a patient as part of an imaging procedure, such as a myocardial perfusion imaging procedure. The one or more physiological parameter sensors may include one or more of an electrocardiographic (EKG) monitor, a blood pressure monitor, a pulse oximeter or the like. Additional and/or alternate physiological parameter sensors may be used within the scope of this disclosure.

The one or more physiological signals received 205 by the processor may include one or more of a blood pressure signal, a heart rate signal, a pulse oximeter signal, a S-T interval signal, and a Q-R-S waveform signal. Additional and/or alternate physiological signals may be received 205 by the processor within the scope of this disclosure.

The processor may compare 210 the one or more physiological signals with one or more threshold values. The comparison 210 may be performed in order to determine whether, for example, a patient is under a sufficient level of cardiac stress during a myocardial perfusion imaging procedure. In an embodiment, a threshold value may be a constant value associated with a particular physiological signal. For example and without limitation, a threshold value may be associated with a blood pressure reading or a heart rate. In an embodiment, a threshold value may correspond to a peak heart rate of about 120 to about 130 beats per minute. In an alternate embodiment, a threshold value may correspond to about 70% of the maximum predicted heart rate based on the age of the patient. Additional and/or alternate physiological information may be compared 210 with a threshold value and/or similar physiological information may be compared with a different threshold value within the scope of this disclosure.

In an embodiment, the one or more threshold values may include at least one threshold value that corresponds to a value of a slope for a physiological signal with respect to time. For example, a procedure may be desired to be performed when a heart rate for an individual is greater than 120 beats per minute and is not increasing. As such, two threshold values may be compared 210 against a heart rate physiological signal. The first threshold value may be satisfied when the patient's heart rate is greater than 120 beats per minute, and the second threshold value may be satisfied when the slope of the patient's heart rate with respect to time is less than or equal to 0. Additional and/or alternate threshold values may be used within the scope of this disclosure, including second derivatives (acceleration), third derivatives (jerk) and/or the like.

In an embodiment, a system may prevent the automatic triggering of the start of the radionuclide when the threshold value is surpassed. This would give additional control over the delivery of the radionuclide to an operator, but with semi-automated assistance. In such an embodiment, an indication that the threshold value was surpassed may be provided to the operator, but manual intervention by the operator would be required to initiate radionuclide delivery.

In an embodiment, the one or more threshold values may be determined 215 based at least in part on patient data. The patient data may correspond to various data relating to the patient, such as the age, gender, height, weight and/or body mass index of the patient. In an embodiment, medical history information may be used as part of the determination 215 of at least one of the one or more threshold values. For example, a patient with a history of heart disease may be placed under less cardiac stress than a patient without such a history. The determination 215 of the one or more threshold values may be automatically performed based on patient data that is identified as part of a pre-screening process. Alternately, the information may be entered manually by an operator.

In an embodiment, the processor may provide 220 an exercise device with one or more regimen signals. The regimen signals may be provided 220 to implement an exercise regimen. The exercise regimen may be designed to result in cardiac stress to the patient (in the case of a myocardial perfusion imaging procedure). In an embodiment, the exercise device may be a treadmill, an exercise bicycle, a stair climber and/or an elliptical aerobic machine. Additional and/or alternate exercise devices may be used within the scope of this disclosure.

In an embodiment, the exercise regimen for the exercise device may be modified based on the one or more physiological signals received by the processor. For example, the one or more physiological signals may be used to determine a current status of the patient. The processor may identify that the patient should exercise more, less or in a different manner in order to achieve a desired set of physiological parameters. In response to such a determination, the processor may modify the regimen signals provided to the exercise device in order to modify the exercise regimen. In an embodiment, the processor may send one or more regimen signals that modify the speed, resistance or inclination of the exercise device. For example, the exercise device may be a treadmill, and the processor may determine that one or both of a speed of the treadmill and an incline of a treadmill should be adjusted. Alternately, if the exercise device is an exercise bicycle, the level of resistance applied to the pedaling subsystem may be increased or decreased as instructed by the processor. Additional and/or alternate methods of adjusting the difficulty or stress to a patient from an exercise device may be performed within the scope of this disclosure.

In an embodiment, the injector device or a separate pharmacological stressor injector device may automatically dispense 225 a pharmacological stressor agent into a patient. The pharmacological stressor agent may be dispensed 225 in response to the one or more physiological signals. In an embodiment, the pharmacological stressor agent may include adenosine. In an embodiment, the pharmacological stressor agent may include dobutamine. For example, the processor may receive one or more physiological signals that identify that the patient is not under a sufficient state of cardiac stress in order to perform a medical procedure, such as myocardial perfusion imaging. As such, the processor may direct the injector device or pharmacological stressor injector device to dispense 225 the pharmacological stressor agent in order to induce additional cardiac stress.

In an alternate embodiment, the processor may receive one or more telemetric signals from an exercise device and direct the stressor device or the pharmacological stressor injector device to dispense 225 a pharmacological stressor agent into the patient in response to the one or more telemetric signals. For example, the one or more telemetric signals may identify one or more of an amount of resistance, a speed and an inclination of the exercise device. The processor may receive the telemetric signals and determine that modifying the exercise regimen for the exercise device would not result in a sufficient level of cardiac stress in the patient. As such, the processor may direct the injector device or the pharmacological stressor injector device to dispense 225 an appropriate amount of the pharmacological stressor agent in the patient in order to induce the appropriate level of cardiac stress for the medical procedure. In an alternate embodiment, an indicator may be provided to a user which directs the user to dispense 225 an appropriate amount of the pharmacological stressor agent. Additional and/or alternate reasons and methods of inducing cardiac stress are included within the scope of this disclosure.

An injector device in communication with the processor may automatically dispense 230 a radionuclide into a patient in response to the one or more physiological signals meeting or exceeding the one or more threshold values. Exceeding a threshold value may refer to moving from a first value for a physiological signal when the patient is in a non-stressed state to a second value for the physiological signal when the patient is in a stressed state, where the threshold value is between the first value and the second value. In an alternate embodiment, the injector device may automatically dispense 230 a radionuclide into a patient in response to the one or more physiological signals satisfying a condition with respect to the one or more threshold values. The condition may include values for each of the one or more physiological signals falling within one or more ranges, being greater than or less than a corresponding threshold value, or the like.

In an embodiment, the injector device may inject or infuse the radionuclide into the patient. In an embodiment, the radionuclide may be technetium-99m (^{99m}Tc). In an alternate embodiment, the radionuclide may be rubidium-82 (⁸²Rb). In an embodiment, a predictive response model may be used to determine when to dispense 230 the radionuclide in response to the one or more physiological signals meeting or exceeding one or more threshold values. In an alternate embodiment, a lookup table may be used to determine when to dispense 230 the radionuclide in response to the one or more physiological signals meeting or exceeding one or more threshold values. In an embodiment, the radionuclide may be dispensed 230 after a time delay in response to the one or more physiological signals meeting or exceeding one or more threshold values. In an alternate embodiment, the radionuclide may be dispensed 230 in response to a user interaction.

In an embodiment, one or more images of the radionuclide within the patient may be captured 235. For example, a nuclear scan may be performed to identify the location and/or movement of the radionuclide through a patient under cardiac stress. The images from the nuclear scan may be captured 235 to identify, for example, whether a coronary artery occlusion is present within the patient without performing invasive surgical tests.

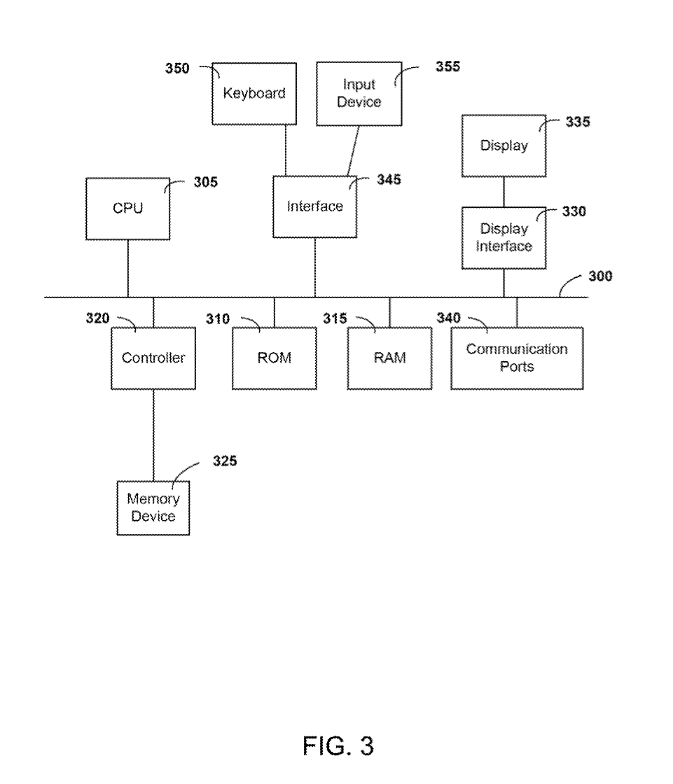

FIG. 3 depicts a block diagram of exemplary internal hardware that may be used to contain or implement program instructions, such as the process steps discussed above in reference to FIG. 2, according to an embodiment. A bus 300 serves as the main information highway interconnecting the other illustrated components of the hardware. CPU 305 is the central processing unit of the system, performing calculations and logic operations required to execute a program. CPU 305, alone or in conjunction with one or more of the other elements disclosed in FIGS. 1 and 3, is an exemplary processing device, computing device or processor as such terms are using in this disclosure. Read only memory (ROM) 310 and random access memory (RAM) 315 constitute exemplary memory devices.

A controller 320 interfaces with one or more optional memory devices 325 to the system bus 300. These memory devices 325 may include, for example, an external or internal DVD drive, a CD ROM drive, a hard drive, flash memory, a USB drive or the like. As indicated previously, these various drives and controllers are optional devices.

Program instructions, software or interactive modules for providing the digital marketplace and performing analysis on any received feedback may be stored in the ROM 310 and/or the RAM 315. Optionally, the program instructions may be stored on a tangible computer readable medium such as a compact disk, a digital disk, flash memory, a memory card, a USB drive, an optical disc storage medium, such as a Blu-ray™ disc, and/or other recording medium.

An optional display interface 330 may permit information from the bus 300 to be displayed on the display 335 in audio, visual, graphic or alphanumeric format. Communication with external devices may occur using various communication ports 340. An exemplary communication port 340 may be attached to a communications network, such as the Internet or an intranet.

The hardware may also include an interface 345 which allows for receipt of data from input devices such as a keyboard 350 or other input device 355 such as a mouse, a joystick, a touch screen, a remote control, a pointing device, a video input device and/or an audio input device.

It will be appreciated that various of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which alternatives, variations and improvements are also intended to be encompassed by the following claims.

What is claimed is:

1. A system for dispensing a radionuclide, comprising:
a processor;
one or more physiological parameter sensors, wherein each physiological parameter sensor is in operable communication with the processor;
an injector device in operable communication with the processor; and
a non-transitory, computer-readable storage medium in operable communication with the processor, wherein the computer-readable storage medium contains one or more programming instructions that, when executed, cause the processor to:
receive one or more physiological signals from the one or more physiological parameter sensors,
compare the one or more physiological signals with one or more threshold values, and
cause the injector device to dispense a radionuclide in response to the one or more physiological signals meeting or exceeding the one or more threshold values.

2. The system of claim 1, further comprising:
an exercise device in operable communication with the processor, and
wherein the computer-readable storage medium further contains one or more programming instructions that, when executed, cause the processor to provide the exercise device with one or more regimen signals used to implement an exercise regimen.

3. The system of claim 2, wherein the one or more programming instructions that, when executed, cause the processor to provide the exercise device with one or more regimen signals further comprise one or more programming instructions that, when executed, cause the processor to modify the exercise regimen for the exercise device based on the one or more physiological signals.

4. The system of claim 2, wherein the exercise device comprises a treadmill, an exercise bicycle, a stair climber or an elliptical aerobic machine, and
wherein the one or more programming instructions further comprise one or more programming instructions that, when executed, cause the processor to receive one or more telemetric signals from the exercise device.

5. The system of claim 4, wherein the one or more telemetric signals comprise one or more of an amount of resistance, a speed and an inclination of the exercise device.

6. The system of claim 1, further comprising:
a pharmacological stressor injector device in operable communication with the processor, and
wherein the computer-readable storage medium further contains one or more programming instructions that, when executed, cause the processor to cause the pharmacological stressor injector device to dispense a pharmacological stressor agent.

7. The system of claim 6, wherein the one or more programming instructions that, when executed, cause the processor to cause the pharmacological stressor injector device to dispense a pharmacological stressor agent further comprise one or more programming instructions that, when executed, cause the processor to cause the pharmacological stressor injector device to dispense a pharmacological stressor agent in response to the one or more physiological signals.

8. The system of claim 6, wherein the pharmacological stressor agent comprises adenosine or dobutamine.

9. The system of claim 1, wherein the computer-readable storage medium further contains one or more programming instructions that, when executed, cause the processor to determine the one or more threshold values based at least on patient data.

10. The system of claim 9, wherein the patient data comprises one or more of an age, a gender, a height, a weight, and a body mass index of a patient.

11. The system of claim 1, wherein the one or more physiological signals comprise one or more of a blood pressure signal, a heart rate signal, a pulse oximeter signal, a S-T interval signal, and a Q-R-S waveform signal.

12. The system of claim 1, wherein the one or more physiological parameter sensors comprise an electrocardiographic (EKG) monitor, a blood pressure monitor, or a pulse oximiter.

13. The system of claim 1, wherein the radionuclide comprises technetium-99m (^{99m}Tc), rubidium-82 (⁸²Rb), thallium-201, iodine-123 (¹²³I), iodine-131 (¹³¹I), ¹⁸F-deoxyglucose (FDG), ¹³N-ammonia, or ¹¹C-choline.

14. A method of dispensing a radionuclide, comprising:
receiving, by a processor, one or more physiological signals from one or more physiological parameter sensors;
comparing, by the processor, the one or more physiological signals with one or more threshold values; and
automatically dispensing, by an injector device in communication with the processor, a radionuclide into a patient in response to the one or more physiological signals meeting or exceeding the one or more threshold values.

15. The method of claim 14, further comprising:
capturing one or more images of the radionuclide within the patient.

16. The method of claim 14, further comprising:
providing an exercise device with one or more regimen signals used to implement an exercise regimen.

17. The method of claim 16, wherein the exercise device comprises a treadmill, an exercise bicycle, a stair climber or an elliptical aerobic machine.

18. The method of claim 16, wherein providing an exercise device with one or more regimen signals further comprises modifying the exercise regimen for the exercise device based on the one or more physiological signals.

19. The method of claim 14, further comprising:
determining the one or more threshold values based at least on patient data, wherein the patient data comprises data associated with the patient.

20. The method of claim 19, wherein the patient data comprises one or more of an age, a gender, a height, a weight, and a body mass index of a patient.

21. The method of claim 14, further comprising:
automatically dispensing a pharmacological stressor agent into a patient.

22. The method of claim 21, wherein automatically dispensing a pharmacological stressor agent comprises dispensing a pharmacological stressor agent into the patient in response to the one or more physiological signals.

23. The method of claim 21, wherein automatically dispensing a pharmacological stressor agent comprises:
receiving one or more telemetric signals from an exercise device; and
dispensing a pharmacological stressor agent into the patient in response to the one or more telemetric signals.

24. The method of claim 23, wherein the one or more telemetric signals comprise one or more of an amount of resistance, a speed and an inclination of the exercise device.

25. The method of claim 21, wherein the pharmacological stressor agent comprises adenosine or dobutamine.

26. The method of claim 14, wherein the one or more physiological signals comprise one or more of a blood pressure signal, a heart rate signal, a pulse oximeter signal, a S-T interval signal, and a Q-R-S waveform signal.

27. The method of claim 14, wherein the one or more physiological parameter sensors comprise an electrocardiographic (EKG) monitor, a blood pressure monitor, or a pulse oximeter.

28. The method of claim 14, wherein the radionuclide comprises technetium-99m (^{99m}Tc), rubidium-82 (⁸²Rb), thallium-201, iodine-123 (¹²³I), iodine-131 (¹³¹I), ¹⁸F-deoxyglucose (FDG), ¹³N-ammonia, or ¹¹C-choline.
